(19) United States
(12) Patent Application Publication
(10) Pub. No.: US 2021/0259779 A1
(43) Pub. Date: Aug. 26, 2021

(54) MULTI-CAMERA USER INTERFACE DEVICE CALIBRATION AND TRACKING

(71) Applicant: Verb Surgical Inc., Santa Clara, CA (US)

(72) Inventors: Bernhard A. Fuerst, Sunnyvale, CA (US); Alexander Barthel, Sunnyvale, CA (US)

(21) Appl. No.: 16/795,909

(22) Filed: Feb. 20, 2020

(51) Int. Cl. A61B 34/20; A61B 34/35; G06T 7/80; G06T 7/00; G06T 7/33

(52) U.S. Cl. CPC A61B 34/20; A61B 34/35; G06T 7/80; G06T 7/0012; G06T 2207/10068; A61B 2034/2057; A61B 2034/2065; G06T 2207/30208; G06T 7/33

(57) ABSTRACT

Surgical robotic system includes a surgical robotic arm, a handheld user interface device (UID) having a camera. Images from the camera are processed to detect a marker in the user environment. A pose of the handheld UID is determined based on the detected marker. A movement of the surgical robotic arm is effected based on the pose of the handheld UID.

### TECHNICAL FIELD

This disclosure relates generally to the field of surgical robotics and, more particularly, to calibration and tracking of a user interface device (UID) for use with a surgical robotic system.

### BACKGROUND

Minimally-invasive surgery (MIS), such as laparoscopic surgery, involves techniques intended to reduce tissue damage during a surgical procedure. For example, laparoscopic procedures typically involve creating a number of small incisions in the patient (e.g., in the abdomen), and introducing one or more tools and at least one endoscopic camera through the incisions into the patient. The surgical procedures are then performed by using the introduced tools, with the visualization aid provided by the camera.

Generally, MIS provides multiple benefits, such as reduced patient scarring, less patient pain, shorter patient recovery periods, and lower medical treatment costs associated with patient recovery. In some embodiments, MIS may be performed with surgical robotic systems that include one or more robotic arms for manipulating surgical instruments based on commands from an operator.

An operator of the surgical robotic system can use a user interface device (UID) to control the surgical robotic arms. A surgical robotic arm can multiple joints having many degrees of freedom, resulting in a vast number of unique poses. During surgery, control of the surgical robotic arm within a workspace is crucial.

A UID that provides a wide range of spatial control is beneficial for controlling surgical robotic arms. Although UIDs exist for non-surgical applications such as, for example, gaming consoles, personal computers, and remote control vehicles, these UIDs might not be suitable for a surgical robotic system due. Given the nature of surgery and potential of harm to a patient, precision and reliability of a UID that is used to control a surgical robotic arm during surgery is of high importance.

### SUMMARY

Generally, a user interface device (UID) can have one or more cameras to track the UID. The pose of the UID (e.g., with six degrees of freedom) is tracked based on detection of one or more markers with fixed position and orientation in a user environment. A calibration can performed with the UID to determine and optimize transformations between each camera of the UID. These transformations map how one camera is arranged on the UID relative to another camera on the UID. The calibration can also determine and optimize transformations between markers that are fixed to an area surrounding the user.

If multiple markers are tracked by multiple cameras, an optimization can be performed to reduce error, thereby improving the determination of the UID pose. During calibration, the marker to marker transformations and camera to camera transformations can be optimized together, also reducing error and improving accuracy of UID tracking. The UID pose can be determined with six degrees of freedom, thus providing a wide range of spatial inputs, including rotation of the UID in different axis. A surgical robotic arm can be moved based on movement of the UID.

### BRIEF DESCRIPTION OF THE DRAWINGS

FIG. 1 is a pictorial view of an example surgical robotic system in an operating arena.

FIG. 2 shows a system diagram of a surgical robotic system with a multi-camera UID.

FIG. 3A and FIG. 3B shows a UID according to one embodiment.

FIG. 4 shows synchronized cameras of a UID according to one embodiment.

FIG. 5 shows calibration transformations used with a multi-camera UID according to one embodiment.

FIG. 6 shows a matrix representation of a transformation.

FIG. 7 shows a process for tracking of a UID according to one embodiment.

FIG. 8 shows a process for calibration and tracking of a UID according to one embodiment.

### DETAILED DESCRIPTION

Non-limiting examples of various aspects and variations of the invention are described herein and illustrated in the accompanying drawings.

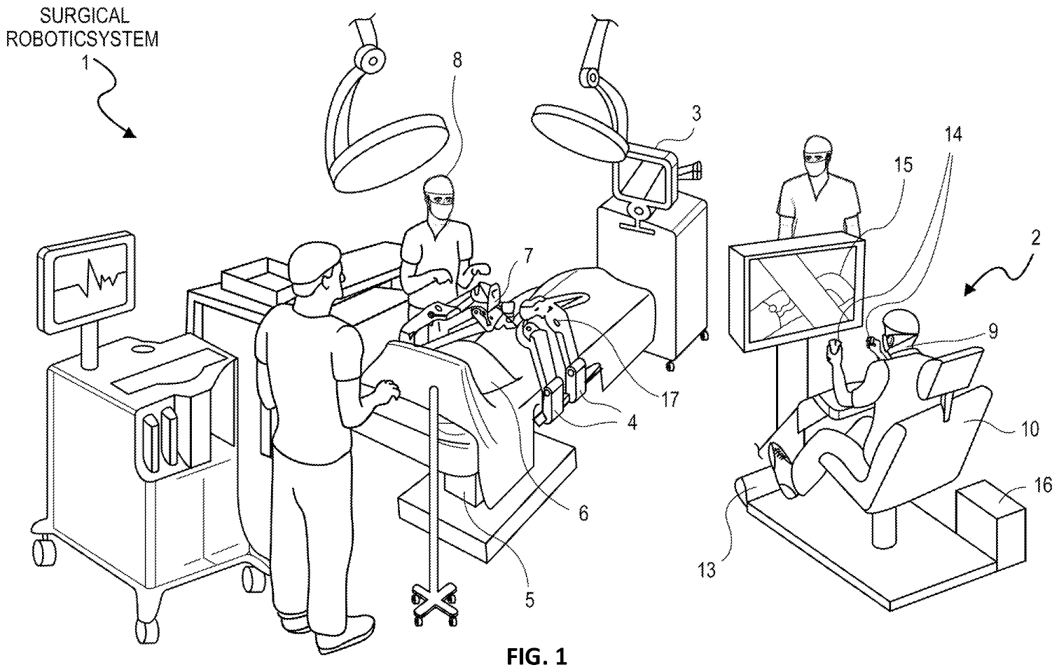

Referring to FIG. 1, this is a pictorial view of an example surgical robotic system 1 in an operating arena. The robotic system 1 includes a user console 2, a control tower 3, and one or more surgical robotic arms 4 at a surgical robotic platform 5, e.g., a table, a bed, etc. The system 1 can incorporate any number of devices, tools, or accessories used to perform surgery on a patient 6. For example, the system 1 may include one or more surgical tools 7 used to perform surgery. A surgical tool 7 may be an end effector that is attached to a distal end of a surgical arm 4, for executing a surgical procedure.

Each surgical tool 7 may be manipulated manually, robotically, or both, during the surgery. For example, the surgical tool 7 may be a tool used to enter, view, or manipulate an internal anatomy of the patient 6. In one embodiment, the surgical tool 7 is a grasper that can grasp tissue of the patient. The surgical tool 7 may be controlled manually, by a bedside operator 8; or it may be controlled robotically, via actuated movement of the surgical robotic arm 4 to which it is attached. The robotic arms 4 are shown as a table-mounted system, but in other configurations the arms 4 may be mounted in a cart, ceiling or sidewall, or in another suitable structural support.

Generally, a remote operator 9, such as a surgeon or other operator, may use the user console 2 to remotely manipulate the arms 4 or the attached surgical tools 7, e.g., teleoperation. The user console 2 may be located in the same operating room as the rest of the system 1, as shown in FIG. 1. In other environments, however, the user console 2 may be located in an adjacent or nearby room, or it may be at a remote location, e.g., in a different building, city, or country. The user console 2 may comprise a seat 10, foot-operated controls 13, one or more handheld user input devices, UID 14, and at least one user display 15 that is configured to display, for example, a view of the surgical site inside the patient 6. In the example user console 2, the remote operator 9 is sitting in the seat 10 and viewing the user display 15 while manipulating a foot-operated control 13 and a handheld UID 14 in order to remotely control the arms 4 and the surgical tools 7 (that are mounted on the distal ends of the arms 4.)

In some variations, the bedside operator 8 may also operate the system 1 in an “over the bed” mode, in which the beside operator 8 (user) is now at a side of the patient 6 and is simultaneously manipulating a robotically-driven tool (end effector as attached to the arm 4), e.g., with a handheld UID 14 held in one hand, and a manual laparoscopic tool. For example, the bedside operator's left hand may be manipulating the handheld UID to control a robotic component, while the bedside operator's right hand may be manipulating a manual laparoscopic tool. Thus, in these variations, the bedside operator 8 may perform both robotic-assisted minimally invasive surgery and manual laparoscopic surgery on the patient 6.

During an example procedure (surgery), the patient 6 is prepped and draped in a sterile fashion to achieve anesthesia. Initial access to the surgical site may be performed manually while the arms of the robotic system 1 are in a stowed configuration or withdrawn configuration (to facilitate access to the surgical site.) Once access is completed, initial positioning or preparation of the robotic system 1 including its arms 4 may be performed. Next, the surgery proceeds with the remote operator 9 at the user console 2 utilizing the foot-operated controls 13 and the UIDs 14 to manipulate the various end effectors and perhaps an imaging system, to perform the surgery. Manual assistance may also be provided at the procedure bed or table, by sterile-gowned bedside personnel, e.g., the bedside operator 8 who may perform tasks such as retracting tissues, performing manual repositioning, and tool exchange upon one or more of the robotic arms 4. Non-sterile personnel may also be present to assist the remote operator 9 at the user console 2. When the procedure or surgery is completed, the system 1 and the user console 2 may be configured or set in a state to facilitate post-operative procedures such as cleaning or sterilization and healthcare record entry or printout via the user console 2.

In one embodiment, the remote operator 9 holds and moves the UID 14 to provide an input command to move a robot arm actuator 17 in the robotic system 1. The UID 14 may be communicatively coupled to the rest of the robotic system 1, e.g., via a console computer system 16. The UID 14 can generate spatial state signals corresponding to movement of the UID 14, e.g. position and orientation of the handheld housing of the UID, and the spatial state signals may be input signals to control a motion of the robot arm actuator 17. The robotic system 1 may use control signals derived from the spatial state signals, to control proportional motion of the actuator 17. In one embodiment, a console processor of the console computer system 16 receives the spatial state signals and generates the corresponding control signals. Based on these control signals, which control how the actuator 17 is energized to move a segment or link of the arm 4, the movement of a corresponding surgical tool that is attached to the arm may mimic the movement of the UID 14. Similarly, interaction between the remote operator 9 and the UID 14 can generate for example a grip control signal that causes a jaw of a grasper of the surgical tool 7 to close and grip the tissue of patient 6.

The surgical robotic system 1 may include several UIDs 14, where respective control signals are generated for each UID that control the actuators and the surgical tool (end effector) of a respective arm 4. For example, the remote operator 9 may move a first UID 14 to control the motion of an actuator 17 that is in a left robotic arm, where the actuator responds by moving linkages, gears, etc., in that arm 4. Similarly, movement of a second UID 14 by the remote operator 9 controls the motion of another actuator 17, which in turn moves other linkages, gears, etc., of the robotic system 1. The robotic system 1 may include a right arm 4 that is secured to the bed or table to the right side of the patient, and a left arm 4 that is at the left side of the patient. An actuator 17 may include one or more motors that are controlled so that they drive the rotation of a joint of the arm 4, to for example change, relative to the patient, an orientation of an endoscope or a grasper of the surgical tool 7 that is attached to that arm. Motion of several actuators 17 in the same arm 4 can be controlled by the spatial state signals generated from a particular UID 14. The UIDs 14 can also control motion of respective surgical tool graspers. For example, each UID 14 can generate a respective grip signal to control motion of an actuator, e.g., a linear actuator, that opens or closes jaws of the grasper at a distal end of surgical tool 7 to grip tissue within patient 6.

In some aspects, the communication between the platform 5 and the user console 2 may be through a control tower 3, which may translate user commands that are received from the user console 2 (and more particularly from the console computer system 16) into robotic control commands that transmitted to the arms 4 on the robotic platform 5. The control tower 3 may also transmit status and feedback from the platform 5 back to the user console 2. The communication connections between the robotic platform 5, the user console 2, and the control tower 3 may be via wired or wireless links, using any suitable ones of a variety of data communication protocols. Any wired connections may be optionally built into the floor or walls or ceiling of the operating room. The robotic system 1 may provide video output to one or more displays, including displays within the operating room as well as remote displays that are accessible via the Internet or other networks (e.g., the robotic system 1 can include one or more endoscopic cameras that provide video output or other suitable image data to the displays). The video output or feed may also be encrypted to ensure privacy and all or portions of the video output may be saved to a server or electronic healthcare record system.

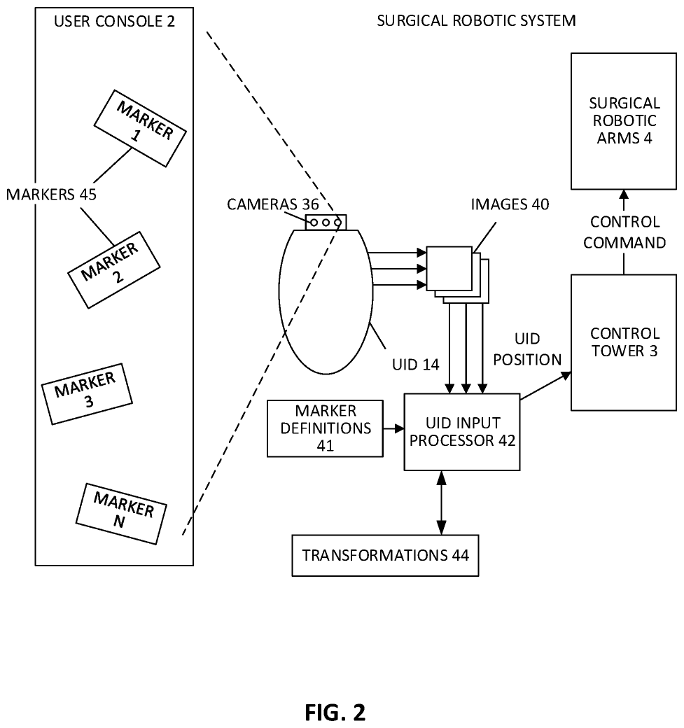

Referring to FIG. 2, an overview of a surgical robotic system (like that described in FIG. 1) is shown according to some embodiments. The system includes a UID 14, and features for calibration and position tracking of the UID. The hardware setup, calibration, and optical tracking with one or more cameras integrated in the UID can be used for teleoperation with a surgical robot. Such a system can be integrated with a user console as shown in FIG. 1. The system and method enlarges the workspace and makes successful tracking of the UID possible for a wide range of movement and poses of the UID. Such a system reduces loss of line-of-sight to optical markers that are fixed at positions in the user's environment.

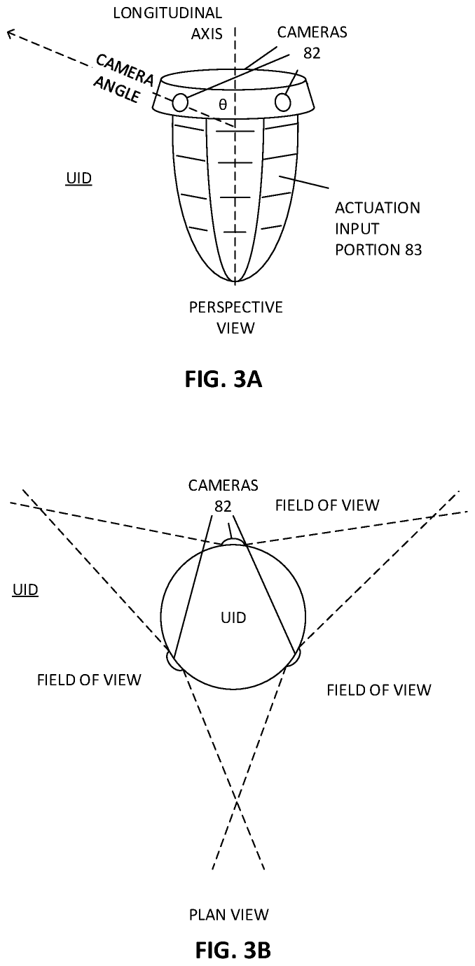

Handheld UID 14 has one or more cameras 36 that are fixed to the UID (e.g., mounted upon), arranged in a manner that sufficiently captures a user's environment. An example UID is shown in FIG. 3A and FIG. 3B. FIG. 3A shows a perspective view of a UID having an actuation input portion 83. This portion of the UID can include a squeezable bulb. When the user squeezes the bulb, this can change an activation state which can be communicated to the UID input processor, similar to a button or a mouse click. In some embodiments, the UID can include buttons, capacitive or resistive touch sensors, or other input means. As shown in FIG. 3B, a plurality of cameras 82 can have a combined field of view that captures a wide view around the UID, for example, a 360-degree view along one or more planes around the UID. It should be understood that each camera can have a cone-shaped field of view. The field of views can overlap, which can improve sel5f-monitoring and optimization, for example, when a marker is detected by more than one camera.

In one embodiment, the UID has three or more cameras. In another embodiment, the UID has exactly three cameras. The cameras are faced outward at an angle θ with respect to a longitudinal axis of the UID that passes between the cameras through a center of UID (e.g., through a center of the actuation input portion of the UID). The angle θ is greater than 0 (for example, between 20 and 90 degrees).

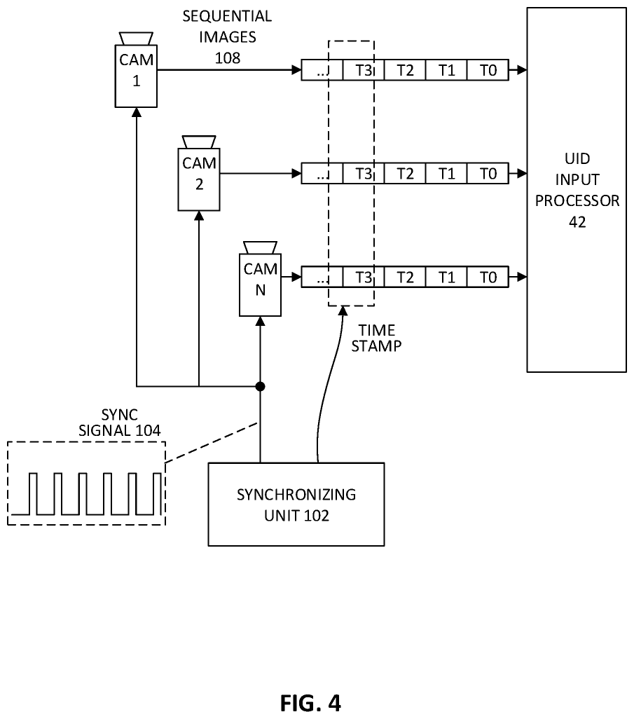

Referring back to FIG. 2, in embodiments where the UID has a plurality of cameras, each of the plurality of cameras can have synchronized shutters to generate time-synchronized images 40. These images are processed by the UID input processor 42 to recognize and track one or more of the markers 45. To further illustrate the synchronizing features, FIG. 4 shows a synchronizing unit 42 that generates a synchronization signal 104. Each camera can open and close its respective shutter in response to the synchronization signal, thereby generating a sequence of images. For example, if the signal is a square wave, the shutters can open and close in response to a rising and falling edge of signal. The signal can be analog or digital and is not limited to a square wave, although shown as such for simplicity.

Images 108 can be captured sequentially by each camera. When multiple cameras are mounted on the UID, the images from the cameras are time-synchronized from one camera to another. The synchronization unit can add a timestamp to each frame of each image sequence so that the UID input processor 106 can analyze images from different cameras that were captured at the same time. This reduces reprojection error in determining UID position based on images from multiple cameras by providing an accurate visual representation of the environment around a user at a given time. Reprojection error is a geometric error corresponding to the image distance between a projected point and a measured one. It is a quantification of how close an estimate of a 3D point recreates the point's true projection.

Referring back to FIG. 2, UID processor 42 can process the time-synchronized images captured from each of the plurality of cameras to recognize at least one of a plurality of markers 45 having a fixed position in a surrounding environment. In some embodiments, the markers are fixed in different positions on a user console 2. A marker can include one of: a plurality of infrared light sources, or a plurality of visible light sources. The lights on each source can form a unique pattern (blobs or dots) so that each marker can be recognized. In some aspects, the light patterns are random and unique, so that even if some of the lights of a marker are blocked, a marker's position and orientation in (having six degrees of freedom) can still be determined.

Marker definitions 41 describe geometry of each pattern of each marker. These definitions can be stored in electronic memory and referenced by the UID input processor to recognize and track the marker poses. The marker definition can include distances and transformations between each blob dot. Marker recognition can be performed by the UID position processor using known computer vision techniques, such as blob detection to recognize the markers and the pose of each marker in the images from the cameras. It should be understood that a pose can include a position and orientation of an object such as a UID, marker, or camera. The pose (e.g., position and orientation) can be described by coordinates. Coordinates can be numeric or symbolic representations of the position and/or orientation an object.

When a marker of the plurality of markers is recognized in one of the time-synchronized images 40 captured by one of the UID cameras, the UID processor can determine a marker to camera transformation that maps a pose of the marker to a pose of the camera that captured the marker. The marker to camera transformation can be determined based on a) the marker definitions, which describes the geometry of each pattern on each marker, and b) the tracked marker in the UID camera image, through known computer vision techniques such as blob detection and perspective projection. Blob detection can recognize and track a marker, while perspective proj ection extrapolates the camera's pose based on the pose of the marker.

The UID processor can determine a pose of the handheld UID based on the pose of the camera. The UID pose can be described with six degrees of freedom (rotation and translation along X, Y, and Z coordinates). The pose of the UID can be represented to align with the longitudinal axis of the UID or any axis, imaginary origin, and/or point relative to the one or more cameras of the UID. The UID position can be processed by a control tower 3 to generate a control command that can modify a pose of at least one of the plurality of surgical robotic arms, based on the pose of the handheld UID. It should be noted that, although shown as a single handheld UID, the system can include multiple UIDs. For example, a user can hold a UID on each hand to control movement of surgical robotic arms.

During use, the UID input processor 42 can improve UID pose determination when multiple cameras capture the same marker, or multiple markers are captured by multiple cameras. When multiple cameras see the same marker, or different markers, these errors can be minimized and spread among the measurements to improve accuracy of the UID pose.

A plurality of transformations 44 are used to improve UID pose determination. The transformations include marker to marker transformations that each map coordinates of one of the plurality of markers to another of the plurality of markers. The transformations also include a plurality of camera to camera transformations that each map coordinates of one of the plurality of cameras to another of the plurality of cameras. The transformations can be determined during an initial calibration phase and then adjusted during use. All of the transformations can be optimized together to minimize and spread error over transformations between each marker and between each camera. Transformations can also include transformation between one or more markers and the user console, and a transformation between one or more cameras and the UID.

It should be understood that the processor 42, transformations 44, and marker definitions 41 can be integral to the control tower 3 or the console computer system 16, or a separate controller. The architecture of hardware and software can vary without departing from the scope of the present disclosure.

FIG. 5 illustrates how the calibration transformations (e.g., transformations 44 as discussed in FIG. 2) can be used to improve UID pose calculation. For example, the UID position processor can recognize and track a first marker image (Marker 2) captured by a first camera (Cam 1) of the UID. In addition, the first marker (Marker 2) is recognized and tracked in images captured by a second camera. Tracking here means that a pose (having location and orientation) of the marker is determined and monitored over multiple frames. The images of the cameras are all time-synchronized, so that image sequences from the cameras accurately represent the surroundings of the user at the same given time.

The UID can determine a marker to camera transformation (^{M2}T_{C1}) that maps a pose of the Marker 2 to a pose of the first camera (Cam 1), and a marker to camera transformation (^{M2}T_{C2}) that maps a pose of Marker 2 to a pose of the second camera (Cam 2). The UID can then optimize the pose of the UID based on a) ^{M2}T_{C1}, b) ^{M2}T_{C2}, and c) a camera to camera transformation (^{C1}T_{C2}) that maps the pose of the first camera to the pose of the second camera.

For example, if there is a discrepancy (e.g., reprojection error) between the pose of the marker as tracked in images by the first camera and those tracked in images by the second camera, this error can be minimized to optimize the UID pose determination. The process of minimizing the reprojection error can include determining a least squares solution. The method of least squares is an approach in regression analysis that approximates the solution of overdetermined systems (sets of equations in which there are more equations than unknowns) by minimizing the sum of the squares of the residuals (e.g., reprojection error) made in the results of every single equation. In this case, the least squares solution will spread the reprojection error across ^{M2}T_{C1}, ^{M2}T_{C2}, and ^{C1}T_{C2}, resulting in an optimized UID pose. Other equivalent optimization techniques, other than least squares, can be used. In such a manner if two or more cameras capture the same marker across the same time stamp, then even if a camera position has some error, the system can minimize this error, thereby providing a more accurate UID pose.

Similarly, if multiple markers are captured by multiple cameras, marker to marker calibrations can further be used to distribute error and optimize the UID pose. For example, if Cam 3 captures Marker 3, and Cam 1 captures Marker 1, then the pose of the UID can be determined be determined through optimization that minimizes error between ^{C1}T_{M1}, ^{M3}T_{C3}, ^{M1}T_{M3}, and ^{C1}T_{C3}. It should be understood, that every transformation can be bi-directional, where a reversal of direction between cameras, markers, and cameras and markers can be performed as an inverse of the transformation.

The examples discussed above show how transformations that are associated with multiple cameras that capture multiple markers can be optimized. The transformations map every marker to every other marker, and every camera to every other camera. Thus, the system can recognize and track a plurality of markers with a plurality of cameras and determine a marker to camera transformation for each marker/camera pair, to map a pose of the tracked marker to the camera. In this manner, the system can access the marker to marker transformations and camera to camera transformations to determine an optimized pose of the UID, by minimizing a reprojection error between a) the marker to camera transformation of each marker/camera pair, and b) the camera to camera transformations and/or the marker to marker transformations of the cameras and markers that are paired.

Such as system reduces error and provides a wide coverage in the user's environment. The markers can be fixed to different areas (for example, on the user console) and when one marker is captured by one camera, a UID pose determination can be made. When multiple markers are captured by multiple cameras, then the UID pose can be optimized.

The marker to marker transformations and camera to camera transformations can be determined during a calibration process, for example, prior to deployment of the system, or after deployment, but prior to use. The system can be recalibrated (adjusting the transformations) at any time, for example, prior to use, or between procedures. The transformations can be stored in electronic memory.

During calibration, a plurality of markers can be recognized and tracked in multiple sequential images, rather than a single image, to improve reliability of the position tracking of the marker. Transformations can be derived mathematically. For example, looking at FIG. 5, ^{C2}T_{M2} and ^{M2}T_{C1} can be determined through blob detection and other known computer vision techniques, for example, perspective projection. The system can recognize and track a marker through blob detection. Through perspective projection, the system can reference geometry of the marker patterns in a 2D plane. Based on the tracked marker, and reference to the geometry of the marker patterns in a 2D plane, the system can use perspective projection to extrapolate the center of projection, representing a pose of the camera, relative to the marker. A transformation between Cam 1 and Cam 2 can be computed by the following ^{C2}T_{M2} ·^{M2}T_{C1}=^{C2}T_{C1}. Each transformation serves as a ‘link’, such that a pose of the UID can be determined based on a path (formed by connected links) between a detected marker and a UID camera. Multiple paths can be formed by connected links when more than one camera captures one or more markers. When the connected links form two or more paths between any of the detected markers and any of the cameras, the system can optimize the UID pose determination, and recalibrate transformations, so that accuracy is improved after deployment of the system.

The marker to marker transformations and the camera to camera transformations can be optimized by determining a least squares solution that minimizes a reprojection error and distributes the reprojection error across the marker to marker transformations and the camera to camera transformations. Practically, this spreads the error among the different cameras and different markers so that the determination of UID position remains consistent over a wide range of UID position without regard to which camera/marker pair is active.

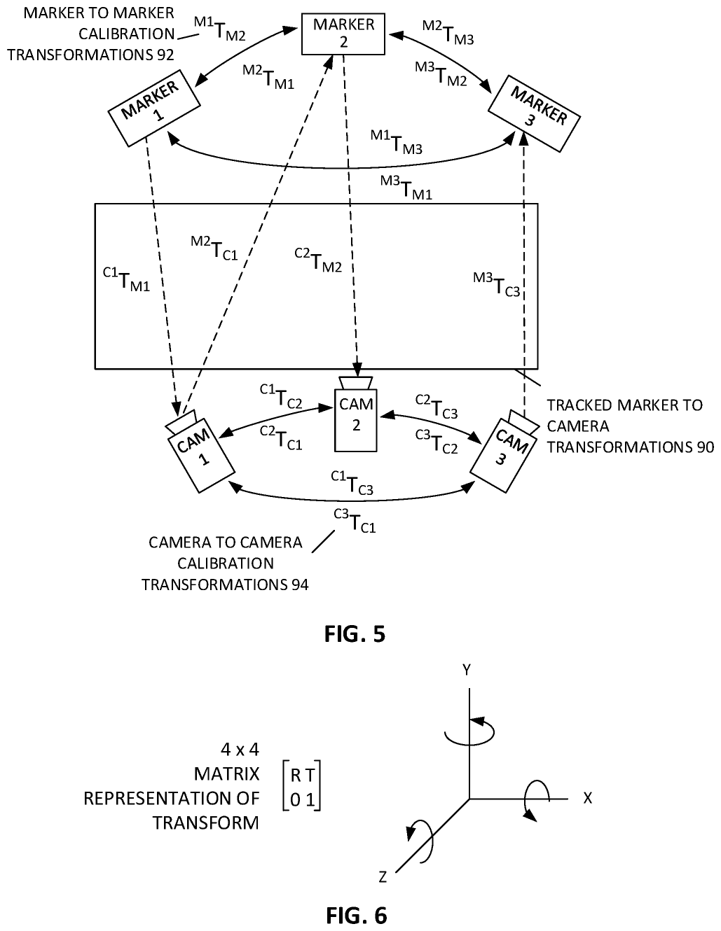

Referring to FIG. 6, all transformations can be represented by matrices, each matrix including rotation ‘R’ and translation ‘T’ of coordinates from one marker to another marker, one camera to another, or between marker and camera. R can be a 3×3 matrix that defines rotation along X, Y, and Z axis. T can be a 1×3 matrix that defines a change in X, Y, and Z coordinates. Thus, the transformation can take the form of a 4×4 transformation matrix.

For example, a transformation matrix such as

$$[ \begin{matrix}1 & 0 & 0 & 0 \\ 0 & cos ∝ & - sin ∝ & 0 \\ 0 & sin ∝ & cos ∝ & 0 \\ 0 & 0 & 0 & 1\end{matrix} ]$$

defines a rotation about the x axis at an angle α. Other values in the transformation matrix can define different rotations about the y and z axis, as is known in the art. Similarly, a transformation such as

$$[ \begin{matrix}1 & 0 & 0 & Δ x \\ 0 & 1 & 0 & Δ y \\ 0 & 0 & 1 & Δ z \\ 0 & 0 & 0 & 1\end{matrix} ]$$

defines a translation in three dimensional space by Δx, Δy, and Δz.

Although calibration is ideally performed once, the calibrated transformations between each camera and between each marker can be adjusted over time. For example, although the cameras are fixed to the UID, shock and vibration of the UID, which can be caused from bumps and drops, can cause the UID camera positions to shift over time. Similarly, although the markers are also in fixed relative positions, transportation of the system can cause changes in the marker positions. The system can adjust the calibration transformations to maintain integrity of the UID tracking.

In some embodiments, a camera to camera transformation can be adjusted when two or more cameras capture the same marker. For example, if a marker is recognized and tracked in images from a first camera and a second camera, marker to camera transformations can be determined between the marker and the first camera and the marker and the second camera. The camera to camera transformations can be adjusted based on a) the marker to camera transformation that maps the pose of the first marker to the pose of the first camera, b) a marker to camera transformation that maps the pose of the first marker to a pose of the second camera, and c) the camera to camera transformations that maps the pose of the first camera to the pose of the second camera. If there is a discrepancy between the two marker to camera transformations, then the difference can be minimized by spreading the error over all the relevant camera to camera transformations and marker to marker transformations.

Camera to camera transformations and marker to marker transformations can also be adjusted when two or more cameras capture different markers. For example, if a first marker is recognized by a first camera and a second marker is recognized by a second camera, the system can determine a marker to camera transformation for each marker/camera pair. A marker to marker transformation maps the first marker pose to the second pose. Similarly, a camera to camera transformation maps the first camera to the second camera. If there is a discrepancy between any of the transformations, then the camera to camera transformations and the marker to marker transformations can be adjusted, based on optimizing the relevant transformations (as discussed in other sections). In this case, the relevant transformations would be the two marker/camera pairs, a camera to camera transformation between the first camera and the second camera, and a marker to marker transformation between the first marker and the second marker. The remaining camera to camera calibration values and marker to marker calibration values can also be adjusted to further spread the error.

Adjusted marker to marker and camera to camera transformations can be stored in electronic memory and used in subsequent calculations, thereby recalibrating the system to adjust for changes. Other measures can be taken when the positions are changed. The system can perform diagnostics and make the user aware that servicing or a replacement part is required. For example, tracking of the markers may indicate that one of the cameras has shifted out of tolerance. Rather than adjust the camera to camera transformations, the system can provide a notification, for example, through a user interface, that the UID is out of calibration, and/or a replacement UID might be needed.

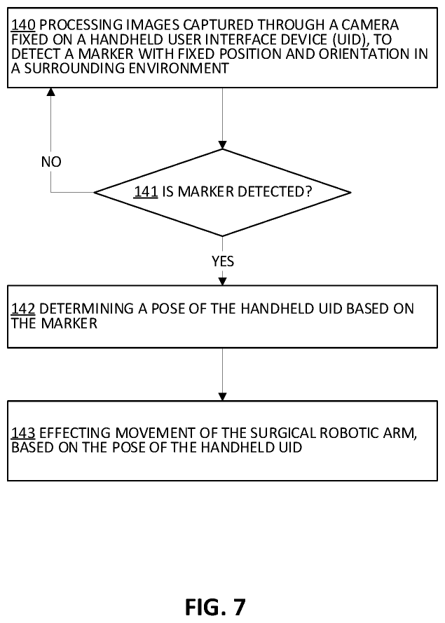

FIG. 7 shows a process for tracking a UID. At block 140, the process includes processing images captured through a camera fixed on a handheld UID to detect a marker having fixed position and orientation in a surrounding environment. In some embodiments, one or more of the marker poses (position and orientation) can be known with respect to the user console. The one or more marker poses can define a point of origin of the coordinate system, which can be anchored to the user console, for one or more handheld UIDs to be tracked relative to the origin of the coordinate system. A user can receive visual feedback on motions of the surgical robotic tools through an endoscope video feed that is shown on a display (e.g., at the user console). The surgical robotic tools can be controlled to mimic pose of the UID, for example, through rotation and translations (directions of movement), thereby providing an intuitive control mechanism.

At block 141, if a marker is detected, then the process proceeds to block 142. Otherwise, the process can continue to process the images to detect whether a marker is present in the images. As discussed, a marker has a detectable pattern (blobs or dots) that the system knows (e.g., stored in marker definitions 41) and can detect through computer vision.

At block 142, the process includes determining a pose of the handheld UID based on the marker. This pose can be determined based on transformations that link a pose of the marker to a pose of the camera. The pose of the camera can define the pose of the UID. For example, the pose of the camera can be known relative to an imaginary or real axis (or origin) of the UID. Thus, as movement of the cameras are tracked relative to the markers, the UID movement is also tracked, because the axis/origin of the UID is known relative to the camera. At block 143, the process includes effecting movement of the surgical robotic arm and/or surgical robotic tool based on the pose of the handheld UID.

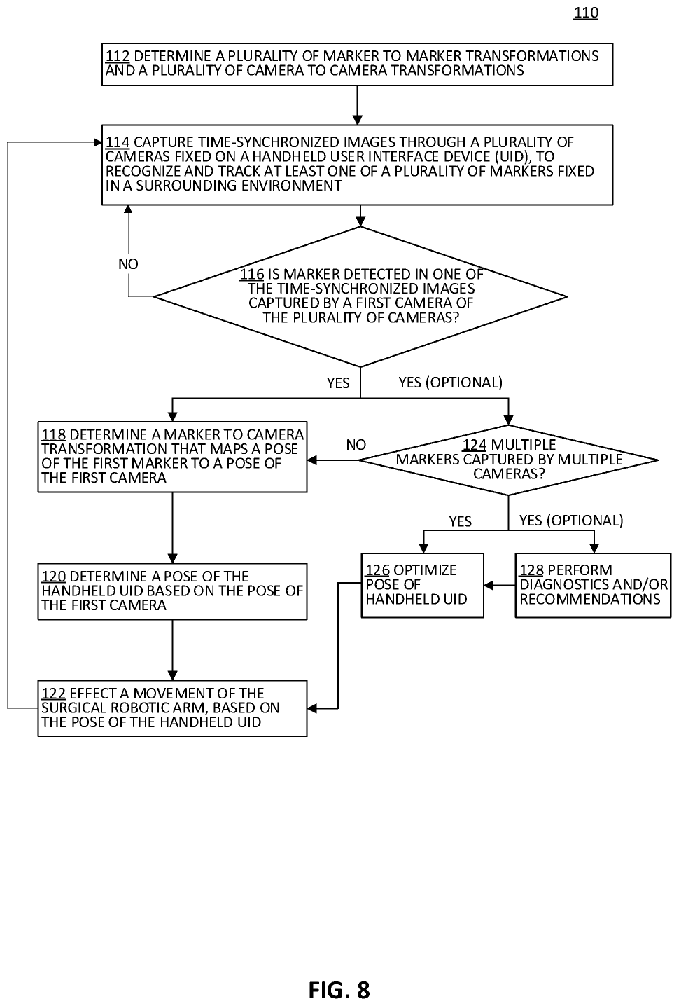

In some embodiments, the handheld UID can have multiple cameras, and multiple markers can be fixed to the environment, as described in FIG. 8. With multiple cameras and multiple markers, accuracy of UID tracking can be improved through optimization. The system can self-calibrate and diagnose problems such as unwanted changes in marker or camera positions after deployment of the system.

FIG. 8 shows a process 110 for multi-camera UID calibration and tracking. At block 112, the process includes determining a plurality of marker to marker transformations and a plurality of camera to camera transformations. For calibration of the marker to marker transformations, pairs of markers must be visible to the cameras of the UID and tracked successfully in multiple frames of a sequence. The transformations are optimized using the tracking results for those frames. Optimization includes minimizing a reprojection error by solving a least squares problem. To determine the pose of the UID using images of multiple cameras, each camera pose must be known relative to each other. Camera to camera transformations can be determined by performing an optimization on the tracking results of each camera using a single image sequence. During calibration, at least two arbitrary markers are in the field of view of at least two cameras at the same time in multiple frames. The transformation between those two cameras are derived.

At block 114, the process includes capturing time-synchronized images through the plurality of cameras fixed on the handheld UID to recognize and track at least one of a plurality of markers having a fixed position and orientation in a surrounding environment. Each of the plurality of cameras have time synchronized shutters to generate the time synchronized images.

At block 116, if a marker is recognized in one of the time-synchronized images captured by a first camera of the plurality of cameras, the process will proceed to block 118. At block 118, the process includes determining a marker to camera transformation that maps a pose of the first marker to a pose of the first camera. At block 120, the process includes determining a pose of the handheld UID based on the pose of the first camera. At block 122, the process includes effecting a movement of the surgical robotic arm (or a surgical robotic tool) based on the pose of the handheld UID.

In some embodiments, the process can improve the accuracy of pose determination through optimization. For example, at block 124, if multiple markers are captured by multiple cameras for a given time stamp, then the process can optionally proceed to block 126 which includes optimizing a pose of the handheld UID. Optimization can be performed by spreading error across the different transformations. For example, as described in other sections, if a second camera captures a second marker, then a least squares solution can be found for transformations between a) the first camera and the second camera, b) the first marker and the second marker, c) the first camera and the first marker, and d) the second marker and the second camera, to minimize a reprojection error of the transformations. A pose of the handheld UID can be determined based on the resulting optimized transformations, thereby calculating an improved or optimized pose of the handheld UID. These optimized transformations can be used to adjust the calibration transformations, which include the marker to marker transformations and the camera to camera transformations. This can be performed, for example, if the transformations appear to have been changed from those previously calculated. In this manner, the transformations, and resulting accuracy of pose determination, can remain accurate even after initial calibration of the system. Based on the optimized transformations, the process can proceed to block 120 to determine a pose of the handheld UID based on the pose of the first camera and/or other cameras that capture a marker.

Additionally or alternatively, the process can proceed to block 128 and perform diagnostics and/or recommendations. For example, based on the captured markers in the multiple cameras, the system can re-determine some of the marker to marker transformations and/or some of the camera to camera transformations. Those transformations that can be re-determined would depend on which markers were detected by which cameras. As mentioned, each transformation can be a link between a marker and another marker, a camera and another camera, or a marker and a camera. Multiple links can form one or more paths linking a sensed marker to a camera, from which pose of the UID can be determined. If different paths yield different results of the UID pose, then the transformations can be re-calculated (e.g., optimized) to spread error across the transformations. Further, these transformations can indicate that one of the cameras or markers has moved out of place, when compared to how the transformations were previously stored. Diagnostics can be performed to determine which transformations have changed, and how much they have changed. A fault or recommendation can be generated and shown to the user (e.g., through a user interface notification), to indicate that the UID is damaged and should be replaced or that the system should be serviced, and/or that the markers are out of position.

The foregoing description, for purposes of explanation, used specific nomenclature to provide a thorough understanding of the invention. However, it will be apparent to one skilled in the art that specific details are not required in order to practice the invention. Thus, the foregoing descriptions of specific embodiments of the invention are presented for purposes of illustration and description. They are not intended to be exhaustive or to limit the invention to the precise forms disclosed; obviously, many modifications and variations are possible in view of the above teachings. The embodiments were chosen and described in order to best explain the principles of the invention and its practical applications, and they thereby enable others skilled in the art to best utilize the invention and various embodiments with various modifications as are suited to the particular use contemplated.

What is claimed is:

1. A surgical robotic system that includes:
a surgical robotic arm;
a handheld UID having a first camera; and
one or more processors, configured to perform operations, the operations including:
processing images captured by the camera to detect a first marker with fixed position and orientation in a surrounding environment;
determining a pose of the handheld UID based on the first marker; and
effecting movement of the surgical robotic arm, based on the pose of the handheld UID.

2. The surgical robotic system of claim 1, wherein determining the pose of the handheld UID includes determining a marker to camera transformation that maps a pose of the first marker to a pose of the first camera.

3. The surgical robotic system of claim 2, further comprising a plurality of markers, each marker with fixed position and orientation in the surrounding environment, wherein each of a plurality of marker to marker transformations maps coordinates of any one of the plurality of markers to another of the plurality of markers.

4. The surgical robotic system of claim 3, wherein the handheld UID includes a plurality of cameras, and each of a plurality of camera to camera transformations maps coordinates of any one of the plurality of cameras to another of the plurality of cameras.

5. The surgical robotic system of claim 4, further comprising
recognizing a second marker of the plurality of markers in a second camera of the plurality of cameras, and
determining a marker to camera transformation that maps a pose of the second marker to a pose of the second camera, wherein
determining the pose of the UID is further based on minimizing a reprojection error between a) the marker to camera transformation that maps the pose of the first marker to the pose of the first camera, b) the marker to camera transformation that maps the pose of the second marker to the pose of the second camera, and c) one or more of the camera to camera transformations or the marker to camera transformations.

6. The surgical robotic system of claim 4, further comprising:
if the first marker is recognized in a second camera of the plurality of cameras,
determining a marker to camera transformation that maps the pose of the first marker to a pose of the second camera, and
adjusting the camera to camera transformations based on a) the marker to camera transformation that maps the pose of the first marker to the pose of the first camera, b) a marker to camera transformation that maps the pose of the first marker to a pose of the second camera, and c) one of the camera to camera transformations that maps the pose of the first camera to the pose of the second camera.

7. The surgical robotic system of claim 4, further comprising:
if a second marker of the plurality of markers is recognized in the time-synchronized images captured by a second camera of the plurality of cameras, then
determining a marker to camera transformation that maps a pose of the second marker to a pose of the second camera, and
adjusting the camera to camera transformations based on a) the marker to camera transformation that maps the pose of the first marker to the pose of the first camera, b) the marker to camera transformation that maps the pose of the second marker to the pose of the second camera, and c) one of the camera to camera transformations that maps the pose of the first camera to the pose of the second camera.

8. The surgical robotic system of claim 4, further comprising:
if a second marker of the plurality of markers is recognized in a second camera of the plurality of cameras,
determining a marker to camera transformation that maps a pose of the second marker to a pose of the second camera, and
adjusting the marker to marker transformations based on a) the marker to camera transformation that maps the pose of the first marker to the pose of the first camera, b) a marker to camera transformation that maps the pose of the first marker to a pose of the second camera, and c) one of the camera to camera transformations that maps the pose of the first camera to the pose of the second camera.

9. The surgical robotic system of claim 4, wherein the marker to marker transformations and camera to camera transformations are determined during a calibration process and stored in electronic memory.

10. The surgical robotic system of claim 9, wherein during the calibration process,
pairs of the plurality of markers are recognized in multiple sequential images of at least two of the plurality of cameras, and
the marker to marker transformations and the camera to camera transformations are optimized by determining a least squares solution that minimizes a reprojection error and distributing the reprojection error across the marker to marker transformations and the camera to camera transformations.

11. The surgical robotic system of claim 4, wherein the marker to marker transformations and camera to camera transformations are matrices, each matrix including rotation and translation of coordinates from one marker to another marker or one camera to another camera.

12. The surgical robotic system of claim 4, wherein the plurality of cameras are arranged facing away from a longitudinal axis of the handheld UID.

13. The surgical robotic system of claim 12, wherein the longitudinal axis passes through a center of a squeeze actuated bulb of the handheld UID.

14. The surgical robotic system of claim 1, wherein marker detection is performed using blob detection.

15. The surgical robotic system of claim 4, wherein each of the plurality of markers includes one of: a plurality of infrared light sources, or a plurality of visible light sources.

16. A computer implemented method for controlling a surgical robotic arm, comprising:
processing images captured through a first camera mounted on a handheld user interface device (UID);
determining whether a first marker fixed in a surrounding environment is detected;
when the first marker is detected, determining a pose of the handheld UID based on the first marker; and
effecting movement of the surgical robotic arm, based on the pose of the handheld UID.

17. The method of claim 16, wherein determining a pose of the handheld UID includes determining a marker to camera transformation that maps a pose of the first marker to a pose of the first camera.

18. The method of claim 17, wherein a plurality of markers are located in fixed positions in the surrounding environment, and a plurality of marker to marker transformations map coordinates of each of the plurality of markers to each other.

19. The method of claim 18, wherein the handheld UID includes a plurality of cameras and a plurality of camera to camera transformations map coordinates of each of the plurality of cameras to each other.

20. The method of claim 19, further comprising
recognizing a second marker of the plurality of markers in a second camera of the plurality of cameras; and
determining a marker to camera transformation that maps a pose of the second marker to a pose of the second camera,
wherein determining the pose of the UID includes minimizing a reprojection error between a) the marker to camera transformation that maps the pose of the first marker to the pose of the first camera, b) the marker to camera transformation that maps the pose of the second marker to the pose of the second camera, and c) one or more of the camera to camera transformations or the marker to camera transformations.
